(19) United States
(12) Patent Application Publication
(10) Pub. No.: US 2007/0185138 A1
(43) Pub. Date: Aug. 9, 2007

(54) Imidazolopyrimidines used as fungicidal active ingredients

(76) Inventors: Olaf Gebauer, Leverkusen (DE); Herbert Gayer, Monheim (DE); Ulrich Heinemann, Leichlingen (DE); Stefan Herrmann, Langenfeld (DE); Stefan Hillebrand, Neuss (DE); Hans-Ludwig Elbe, Wuppertal (DE); Ronald Ebbert, Nurnberg (DE); Ulrike Wachendorff-Neumann, Neuwied (DE); Peter Dahmen, Neuss (DE); Karl-Heinz Kuck, Langenfeld (DE)

Correspondence Address: BAYER CROPSCIENCE LP, Patent Department, 2 T .W. ALEXANDER DRIVE, RESEARCH TRIANGLE PARK, NC, 27709, US

(21) Appl. No.: 10/588,924

(22) PCT Filed: Feb. 10, 2005
(86) PCT No.: PCT/EP05/01320

§ 371 Date: Mar. 26, 2007

(30) Foreign Application Priority Data
Feb. 13, 2004 (DE) 10 2004 007 076.8

(51) Int. Cl. A61K 31/519

(52) U.S. Cl. 514/259.1; 544/281

(57) ABSTRACT

This invention relates to imidazolopyrimidines of the formula 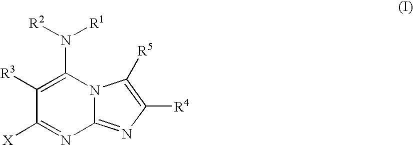 in which
R¹, R², R³, R⁴, R⁵, and X are as defined in the disclosure, to a process for preparing these compounds and to their use for controlling unwanted microorganisms.

The invention relates to imidazolopyrimidines, to a process for the preparation to the use for controlling unwanted microorganisms.

It is already known that certain imidazolopyrimidines have fungicidal properties (see, for example, WO-A 03/022 850).

However, since the ecological and economical demands made on modern fungicides are increasing constantly, for example with respect to activity spectrum, toxicity, selectivity, application rate, formation of residues and favourable manufacture, and there can furthermore be problems, for example, with resistance, there is a constant need to develop novel fungicides which, at least in some areas, have advantages over those of the prior art.

The invention now provides novel imidazolopyrimidines of the formula (I), 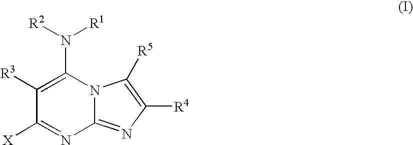
in which
R¹ represents H, R², optionally substituted alkyl, optionally substituted alkenyl, optionally substituted alkynyl, optionally substituted cycloalkyl or represents optionally substituted heterocyclyl,
R² represents an organic radical which contains 3 to 13 carbon atoms and one or more silicon atoms and also, if appropriate, 1 to 3 identical or different heteratoms from the group consisting of oxygen, nitrogen and sulphur and which is unsubstituted or substituted by 1 to 4 identical or different halogens, or
R¹ and R² together with the nitrogen atom to which they are attached represent an optionally substituted heterocyclic ring which contains one or more silicon atoms and/or is substituted by one or more radials R²,
R³ represents optionally substituted aryl, optionally substituted heterocyclyl, optionally substituted alkyl, optionally substituted alkenyl, optionally substituted alkynyl, optionally substituted cycloalkyl, optionally substituted aralkyl, halogen, or an optionally substituted amino group, optionally substituted (C₁-C₈)-alkoxy, optionally substituted (C₁-C₈)-alkythio, optionally substituted (C₆-C₁₀)-aryloxy, optionally substituted (C₆-C₁₀)-arylthio, optionally substituted heterocyclyloxy, optionally substituted (C₆-C₁₀)-aryl-(C₁-C₄)-alkoxy, optionally substituted (C₆-C₁₀)-aryl-(C₁-C₄)-alkylthio, optionally substituted heterocyclyl-(C₁-C₄)-alkoxy, or optionally substituted heterocyclyl-(C₁-C₄)-alkylthio;
R⁴ represents H, halogen, optionally halogen-substituted alkyl or optionally halogen-substituted cycloalkyl;
R⁵ represents H, halogen, optionally halogen-substituted alkyl or optionally halogen-substituted cycloalkyl; and
X represents halogen, cyano, optionally substituted alkyl, optionally substituted alkoxy or optionally substituted phenyl,
   and salts thereof.

Furthermore, it has been found that imidazolopyrimidines of the formula (I-1) 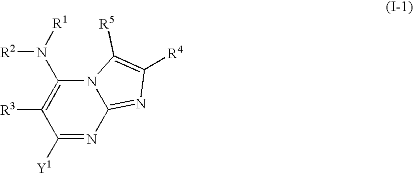
which are derived from the compounds (I) in that X represents Y¹=halogen, can be prepared by:
Process (a) reacting haloimidazolopyrimidines of the formula (II-1), 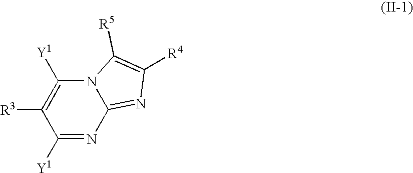
in which
R³, R⁴, R⁵ are as defined above and
Y¹ represents halogen 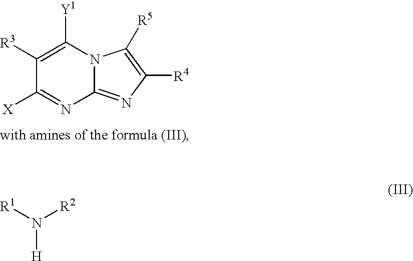
   in which
R¹ and R² are as defined above,
   if appropriate in the presence of a diluent, if appropriate in the presence of an acid acceptor and if appropriate in the presence of a catalyst.

Furthermore, it has been found that imidazolopyrimidines of the formula (I-2), 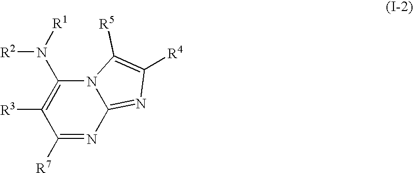
which are derived from the compounds (I) in that X represents R⁷, where R⁷ represents optionally substituted alkyl or optionally substituted phenyl, can be prepared by:
Process (b)
reacting haloimidazolopyrimidines of the formula (II-2), 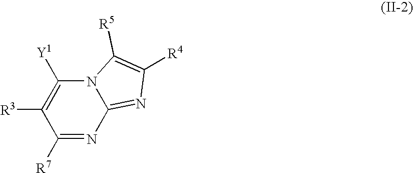
   in which
R³, R⁴, R⁵ are as defined above and
Y¹ represents halogen and
R⁷ represents optionally substituted alkyl or optionally substituted phenyl, with amines of the formula (III), 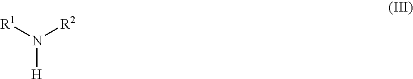
   in which
R¹ and R² are as defined above,
   if appropriate in the presence of a diluent, if appropriate in the presence of an acid acceptor and if appropriate in the presence of a catalyst.

Furthermore, it has been found that imidazolopyrimidines of the formula (I-3), 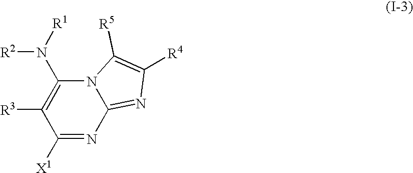
which are derived from the compounds (I) in that X represents X¹=cyano or optionally substituted alkoxy, can be prepared by:
Process (c)
reacting the imidazolopyrimidines of the formula (I-1) 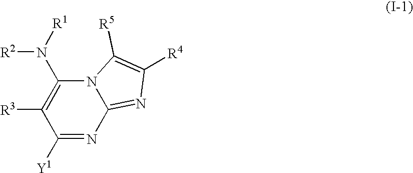
already mentioned analogously to WO-A 02/083677 with a compound of the formula M-X¹ (IX),
   in which the cation M is, for example, ammonium, tetraalkylammonium, an alkali metal, such as lithium, sodium or potassium, or in an alkaline earth metal, such as magnesium, and in which
X¹is cyano, alkoxy or substituted alkoxy, such as haloalkoxy:

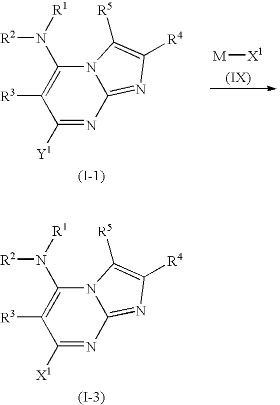

Finally, it has been found that the imidazolopyrimidines of the formula (I) are highly suitable for controlling unwanted microorganisms. In particular, they have high fungicidal activity and can be used both in crop protection and in the protection of materials.

If appropriate, the compounds of the formula (I) according to the invention can be present as mixtures of different possible isomeric forms, in particular of stereoisomers, such as E and Z, threo and erythro, and also optical isomers, such as R and S isomers or atrope isomers, and, if appropriate, also of tautomers.

The formula (I) provides a general definition of the imidazolopyrimidines according to the invention.

Preference is given to compounds of the formula (I) in which
a¹) R³ represents optionally substituted aryl, or
a²) R³ represents optionally substituted heterocyclyl, or
a³) R³ represents optionally substituted alkyl, or
a⁴) R³ represents optionally substituted alkenyl, or
a⁶) R³ represents optionally substituted cycloalkyl, or
a⁷) R³ represents optionally substituted aralkyl, or
a⁸) R³ represents an optionally substituted amino group.

Preference is likewise given to compounds of the formula (I) in which R³ has one of the meanings below:
b¹: a¹, a², a³, a⁴, a⁵, a⁶, a⁷,
b²: a¹, a², a³, a⁴, a⁵, a⁶, a⁸,
b³: a¹, a², a³, a⁴, a⁵, a⁷, a⁸,
b⁴: a¹, a², a³, a⁴, a⁶, a⁷, a⁸,
b⁵: a¹, a², a³, a⁵, a⁶, a⁷, a⁸,
b⁶: a¹, a², a⁴, a⁵, a⁶, a⁷, a⁸,
b⁷: a¹, a³, a⁴, a⁵, a⁶, a⁷, a⁸,
b⁸: a², a³, a⁴, a⁵, a⁶, a⁷, a⁸.

Preference is furthermore given to those compounds of the formula (I) in which one or more symbols have one of the preferred meanings listed below, i.e. in which
R¹ represents H, or
R¹ represents a radical R², or
R¹ represents alkyl having 1 to 6 carbon atoms which may be mono- to pentasubstituted by identical or different substituents from the group consisting of halogen, cyano, hydroxy, alkoxy having 1 to 4 carbon atoms and cycloalkyl having 3 to 8 carbon atoms, or
R¹ represents alkenyl having 2 to 6 carbon atoms which may be mono- to trisubstituted by identical or different substituents from the group consisting of halogen, cyano, hydroxy, alkoxy having 1 to 4 carbon atoms and cycloalkyl having 3 to 8 carbon atoms, or
R¹ represents alkynyl having 3 to 6 carbon atoms which may be mono- to trisubstituted by identical or different substituents from the group consisting of halogen, cyano, alkoxy having 1 to 4 carbon atoms and cycloalkyl having 3 to 8 carbon atoms, or
R¹ represents cycloalkyl having 3 to 8 carbon atoms which may be mono- to trisubstituted by identical or different substituents from the group consisting of halogen and alkyl having 1 to 4 carbon atoms, or
R¹ represents saturated or unsaturated heterocyclyl having 3 to 8 ring members and 1 to 3 heteroatoms, such as nitrogen, oxygen and/or sulphur, where the heterocyclyl may be mono- or disubstituted by halogen, alkyl having 1 to 4 carbon atoms, cyano and/or cycloalkyl having 3 to 8 carbon atoms,
R² represents an aliphatic, saturated or unsaturated group having 1 to 13 carbon atoms and one or more silicon atoms which optionally contains 1 to 3 identical or different heteroatoms from the group consisting of oxygen, sulphur and nitrogen and which is unsubstituted or substituted by 1 to 4 identical or different halogen atoms, or
R¹ and R² together with the nitrogen atom to which they are attached represent a saturated or unsaturated heterocyclic ring having 3 to 8 ring members which contains one or more silicon atoms and/or is substituted by one or more radicals R², where the heterocycle may contain a further nitrogen, oxygen or sulphur atom as ring member and where the heterocycle may furthermore be substituted up to three times by fluorine, chlorine, bromine, alkyl having 1 to 4 carbon atoms and/or haloalkyl having 1 to 4 carbon atoms and 1 to 9 fluorine and/or chlorine atoms;
R³ represents C₁-C₁₀-alkyl, C₂-C₁₀-alkenyl, C₂-C₁₀-alkynyl, C₃-C₈-cycloalkyl or phenyl-C₁-C₁₀-alkyl, where R³ is unsubstituted or partially or fully halogenated and/or optionally carries one to three radicals from the group R^{X}, or C₁-C₁₀haloalkyl which optionally carries one to three radicals from the group R^{X}, and R^{X} represents cyano, nitro, hydroxy, C₁-C₆-alkyl, C₁-C₆-haloalkyl, C₃-C₆-cycloalkyl, C₁-C₆-alkoxy, C₁-C₆-haloalkoxy, C₁-C₆-alkylthio, C₁-C₆-haloalkylthio, C₁-C₆-alkylsulphinyl, C₁-C₆-haloalkylsulphinyl, C₁-C₆-alkylsulphonyl, C₁-C₆-haloalkylsulphonyl, C₁-C₆-alkylamino, di-C₁-C₆-alkylamino, C₂-C₆-alkenyl, C₂-C₆-alkenyloxy, C₂-C₆-alkynyl, C₃-C₆-alkynyloxy and optionally halogenated oxy-C₁-C₄-alkyl-C₁-C₄-alkenoxy, oxy-C₁-C₄-alkenyl-C₁-C₄-alkoxy, oxy-C₁-C₄-alkyl-C₁-C₄-alkyloxy,
R³ represents phenyl which may be mono- to tetrasubstituted by identical or different substituents from the group consisting of
   halogen, cyano, nitro, amino, hydroxy, formyl, carboxy, carbamoyl, thiocarbamoyl;
   in each case straight-chain or branched alkyl, alkoxy, alkylthio, alkylsulphinyl or alkyl-sulphonyl having in each case 1 to 6 carbon atoms;
   in each case straight-chain or branched alkenyl or alkenyloxy having in each case 2 to 6 carbon atoms;
   in each case straight-chain or branched haloalkyl, haloalkoxy, haloalkylthio, haloalkyl-sulphinyl or haloalkylsulphonyl having in each case 1 to 6 carbon atoms and 1 to 13 identical or different halogen atoms;
   in each case straight-chain or branched haloalkenyl or haloalkenyloxy having in each case 2 to 6 carbon atoms and 1 to 11 identical or different halogen atoms;
   in each case straight-chain or branched alkylamino, dialkylamino, alkylcarbonyl, alkyl-carbonyloxy, alkoxycarbonyl, alkylsulphonyloxy, hydroximinoalkyl or alkoximinoalkyl having in each case 1 to 6 carbon atoms in the individual alkyl moieties;
   cycloalkyl having 3 to 8 carbon atoms;
   1,3-propanediyl, 1,4-butanediyl, methylenedioxy (—O—CH₂—O—) or 1,2-ethylenedioxy (—O—CH₂—CH₂—O—), attached in the 2,3 position, where these radicals may be mono- or polysubstituted by identical or different substituents from the group consisting of halogen, alkyl having 1 to 4 carbon atoms and haloalkyl having 1 to 4 carbon atoms and 1 to 9 identical or different halogen atoms;
      or
R³ represents saturated or unsaturated heterocyclyl having 3 to 8 ring members and 1 to 3 heteroatoms from the group consisting of nitrogen, oxygen and sulphur, where the heterocyclyl may be mono- or disubstituted by halogen, alkyl having 1 to 4 carbon atoms, alkoxy having 1 to 4 carbon atoms, alkylthio having 1 to 4 carbon atoms, haloalkoxy having 1 to 4 carbon atoms, haloalkylthio having 1 to 4 carbon atoms, cyano nitro and/or cycloalkyl having 3 to 6 carbon-atoms;
   or
R³ represents C₁-C₈-alkylamino, C₂-C₈-alkenylamino, C₂-C₈-alkynylamino, di-C₁-C₈-alkylamino, di-C₂-C₈-alkenylamino, di-C₂-C₈-alkynylamino, C₂-C₈-alkenyl-(C₂-C₈)-alkynylamino, C₂-C₆-alkynyl-(C₁-C₈)-alkylamino, C₂-C₈-alkenyl-(C₁-C₈)-alkylamino, C₆-C₁₀-arylamino, C₆-C₁₀-aryl-(C₁-C₈)-alkylamino, C₆-C₁₀-aryl-(C₁-C₄)-alkyl-(C₁-C₈)-alkylamino, heterocyclyl-(C₁-C₈)-alkylamino or heterocyclyl-(C₁-C₄)-alkyl-(C₁-C₈)-alkylamino;
R⁴ represents H, halogen, (C₁-C₄)-alkyl which is unsubstituted or substituted by one or more halogen atoms, cyclopropyl which is unsubstituted or substituted by one or more halogen atoms;
R⁵ represents H, halogen, (C₁-C₄)-alkyl which is unsubstituted or substituted by one or more halogen atoms, cyclopropyl which is unsubstituted or substituted by one or more halogen atoms; and
X represents H, fluorine, chlorine, bromnine or CN.

Particular preference is given to those imidazolopyrimidines of the formula (I) in which one or more of the symbols have one of the particularly preferred meanings listed below, i.e. in which
R¹ represents hydrogen, methyl or ethyl;
R² represents a group Y²—Si(OₘCH₃)(OₙCH₃)(OₚY³)
   where m, n and p independently of one another represent 0 or 1;
   Y² represents a bond or alkanediyl, alkenediyl or alkynediyl which are in each case straight-chain or branched, have 1 to 6 and 2 to 6 carbon atoms, respectively, are optionally interrupted by one or two non-adjacent oxygen atoms and are unsubstituted or substituted by 1 to 3 identical or different halogen atoms; and
   Y³ represents straight-chain or branched alkyl or alkenyl having 1 to 5 and 2 to 5 carbon atoms, respectively, optionally interrupted by an oxygen, nitrogen or sulphur atom and unsubstituted or substituted by 1 to 3 identical or different halogen atoms;
R³ represents (C₁-C₈)-alkyl, (C₁-C₈)-cycloalkyl, benzyl or
R³ represents phenyl which may be mono- to trisubstituted by identical or different substituents from the group consisting of
   fluorine, chlorine, bromine, cyano, nitro, formyl, methyl, ethyl, n- or i-propyl, n-, i-, s- or t-butyl, allyl, propargyl, methoxy, ethoxy, n- or i-propoxy, methylthio, ethylthio, n- or i-propylthio, methylsulphinyl, methylsulphinyl, methylsulphonyl, ethylsulphonyl, allyloxy, propargyloxy, trifluoromethyl, trifluoroethyl, difluoromethoxy, trifluoromethoxy, difluorochloromethoxy, trifluoroethoxy, difluoromethylthio, difluorochloromethylthio, trifluoromethylthio, trifluoromethylsulphinyl, trifluoromethylsulphonyl, trichloroethynyloxy, trifluoroethynyloxy, chloroallyloxy, iodopropargyloxy, methylamino, ethylamino, n- or i-propylamino, dimethylamino, diethylamino, acetyl, propionyl, acetyloxy, methoxycarbonyl, ethoxycarbonyl, hydroximinomethyl, hydroximinoethyl, methoximinomethyl, ethoximinomethyl, methoximinoethyl, ethoximinoethyl, cyclopropyl, cyclobutyl, cyclopentyl or cyclohexyl,
   1,3-propanediyl, 1,4-butanediyl, methylenedioxy (—O—CH₂—O—) or 1,2-ethylenedioxy (—O—CH₂—CH₂—O—), attached in the 2,3-position, where these radicals may be mono- or polysubstituted by identical or different substituents from the group consisting of fluorine, chlorine, methyl, ethyl, n-propyl, i-propyl and/or trifluoromethyl,
R³ represents pyridyl which is attached in the 2- or 4-position and may be mono- to tetrasubstituted by identical or different substituents from the group consisting of fluorine, chlorine, bromine, cyano, nitro, methyl, ethyl, methoxy, methylthio, hydroximinomethyl, hydroximinoethyl, methoximinomethyl, methoximinoethyl and/or trifluoromethyl, or
R³ represents pyrimidyl which is attached in the 2- or 4-position and may be mono- to trisubstituted by identical or different substituents from the group consisting of fluorine, chlorine, bromine, cyano, nitro, methyl, ethyl, methoxy, methylthio, hydroximinomethyl, hydroximinoethyl, methoximinomethyl, methoximinoethyl and/or trifluoromethyl, or
R³ represents thienyl which is attached in the 2- or 3-position and may be mono- to trisubstituted by identical or different substituents from the group consisting of fluorine, chlorine, bromine, cyano, nitro, methyl, ethyl, methoxy, methylthio, hydroximinomethyl, hydroximinoethyl, methoximinomethyl, methoximinoethyl and/or trifluoromethyl, or
R³ represents C₁-C₈-alkylamino or di-C₁-C₈-alkylamino, or
R³ represents thiazolyl which is attached in the 2-, 4- or 5-position and may be mono- or disubstituted by identical or different substituents from the group consisting of fluorine, chlorine, bromine, cyano, nitro, methyl, ethyl, methoxy, methylthio, hydroximinomethyl, hydroximinoethyl, methoximinomethyl, methoximinoethyl and trifluoromethyl, or
R³ represents N-piperidinyl, N-tetrazolyl, N-pyrazolyl, N-imidazolyl, N-1,2,4-triazolyl, N-pyrrolyl, or N-morpholinyl which are in each case unsubstituted or mono- or—if possible—polysubstituted by identical or different substituents from the group consisting of fluorine, chlorine, bromine, cyano, nitro, methyl, ethyl, methoxy, methylthio, hydroximinomethyl, hydroximinoethyl, methoximinomethyl, methoximinoethyl and trifluoromethyl,
R⁴ represents H, Cl, F, CH₃, —CH(CH₃)₂ or cyclopropyl;
R⁵ represents H, Cl, F, CH₃, —CH(CH₃)₂ or cyclopropyl; and
X represents H, F, Cl, CN, (C₁-C₄)-alkyl which is unsubstituted or substituted by one or more fluorine or chlorine atoms.

Very particular preference is given to compounds of the formula (I) in which one or more of the symbols have one of the very particularly preferred meanings listed below, i.e. in which
R¹ represents H;
R² represents SiMe₃, SiMe₂Et, SiMe₂CHMe₂, SiMe₂CH₂CHMe₂, SiMe₂CH₂CMe₃, SiMe₂OCHMe₂, SiMe₂OCH₂CHMe₂, CH₂SiMe₃, CH₂SiMe₂Et, CH₂SiMe₂CHMe₂, CH₂SiMe₂CH₂CHMe, CH₂SiMe₂OMe, CH₂SiMe₂OCHMe₂, CH₂SiMe₂OCH₂CHMe₂, CHMeSiMe₃, CHMeSiMe₂OMe, (CH₂)₂SiMe₃, (CH₂)₂SiMe₂Et, (CH₂)₂SiMe₂CHMe₂, (CH₂)₂SiMe₂CMe₃, (CH₂)₂SiMe₂CH₂CHMe₂, (CH₂)₂SiMe₂CH₂CH₂Me, (CH₂)₂SiMe₂CH₂CMe₃, (CH₂)₂SiMe₂OCHMe₂, (CH₂)₂SiMe₂OCH₂CHMe₂, CHMeCH₂SiMe₃, CHMeCH₂SiMe₂Et, CHMeCH₂SiMe₂CH₂CH₂Me, CHMeCH₂SiMe₂CHMe₂, CHMeCH₂SiMe₂CMe₃, CHMeCH₂SiMe₂CH₂CHMe₂, CFMeCH₂SiMe₃, CHMeCH₂CH₂SiMe₂OMe, CHMeCH₂SiMe₂OCHMe₂, CHMeCH₂SiMe₂OCH₂CHMe₂, CH₂CHMeSiMe₃, CH₂CHMeSiMe₂Et, CH₂CHMeSiMe₂CHMe₂, CHMeCHMeSiMe₃, CMe₂CH₂SiMe₃, (CH₂)₃SiMe₃, (CH₂)₃SiMe₂Et, (CH₂)₃SiMe₂CHMe₂, (CH₂)₃SiMe₂CH₂CHMe₂, (CH₂)₃SiMe₂OMe, (CH₂)₃SiMe₂OCHMe₂, (CH₂)₃SiMe₂OCH₂CHMe₂, CHMeCH₂CH₂SiMe₃, CHMeCH₂CH₂SiMe₂Et, CHMeCH₂CH₂SiMe₂CHMe₂, CHMeCH₂CH₂CH₂SiMe₂OMe, CHMeCH₂CH₂SiMe₂OCHMe₂, CMe═CHSiMe₃, CH₂CH₂SiMe₂OMe, —C≡C—SiMe₃, —CH₂—C≡C—SiMe₃ or —CHMe—C≡C—SiMe₃;
R³ represents (C₁-C₆)-alkyl, (C₃-₆)-alkenyl, (C₃-C₆)-alkynyl, (C₃-C₈)-cycloalkyl, where R³ is unsubstituted or substituted by one or more fluorine or chlorine atoms,
   or
R³ represents 2,4- or 2,6-disubstituted phenyl or represents 2-substituted phenyl or represents 2,4,6-trisubstituted phenyl,
R³ represents pyridyl which is attached in the 2- or 4-position and which may be mono- to tetrasubstituted by identical or different substituents from the group consisting of fluorine, chlorine, bromine, cyano, methyl, ethyl, methoxy, methylthio, hydroximinomethyl, hydroximinoethyl, methoximinomethyl, methoximinoethyl and trifluoromethyl, or
R³ represents pyrimidyl which is attached in the 4-position and may be mono- to trisubstituted by identical or different substituents from the group consisting of fluorine, chlorine, bromine, cyano, methyl, ethyl, methoxy, methylthio, hydroximinomethyl, hydroximinoethyl, methoximinomethyl, methoximinoethyl and trifluoromethyl;
R⁴ represents H, —CH₃, —CH(CH₃)₂, Cl or cyclopropyl;
R⁵ represents H, —CH₃, —CH(CH₃)₂, Cl or cyclopropyl; and
X represents fluorine, chlorine, (C₁-C₇)-alkyl or (C₁-C₃)-haloalkyl.

The radical definitions mentioned above may be combined with one another as desired. Moreover, individual definitions may not apply.

The haloimidazolopyrimidines of the formula (II-1) and the formula (II-2) used as starting materials can be synthesized analogously to the preparation processes given in WO-A 03/022850 by process (d) and process (e):

Process (d):

This known process is used to prepare starting materials (II-1) which can be converted by the above process (a) into the imidazolopyrimidines of the formula (I-1) 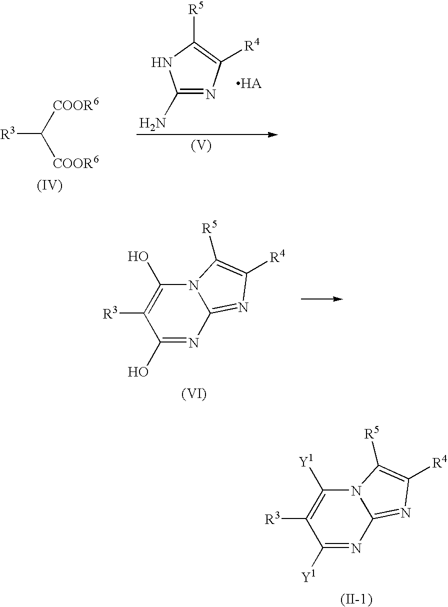
where the symbols are as defined above.
Process (e):

This known process is used to prepare the haloimidazolopyrimidines of the formula (II-2) which can be converted by the above process (b) into the imidazolopyrimidines of the formula (I-2) 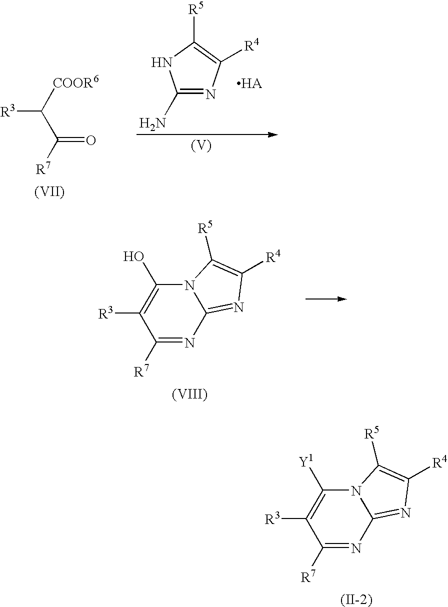
where the symbols are as defined above.

The formula (III) provides a general definition of the amines furthermore required as starting material for carrying out the processes according to the invention. In this formula, R¹ and R² preferably have those meanings which have already been mentioned in connection with the description of the compounds of the formula (I) according to the invention as being preferred for R¹ and R².

The amines of the formula (III) are known. Some of them are commercially available, or they can be prepared, by known methods familiar to the person skilled in the art.

Thus, silylated amines of the formula (IIIa)
H₂N—(CR^{a}R^{b})ₙ—SiR^{c}R^{d}R^{e}
in which
n is a natural number from 0 to 10 and
R^{a}, R^{b}, R^{c}, R^{d} are identical or different radicals H, CH₃ or C₂H₅ (the total number of carbon atoms in R^{a-d} being ≦12),
are generally available by reacting, for example, phthalimide in the presence of a base, such as K₂CO₃, with a haloalkylsilane and cleaving the resulting N-substituted phthalimide with hydrazine:

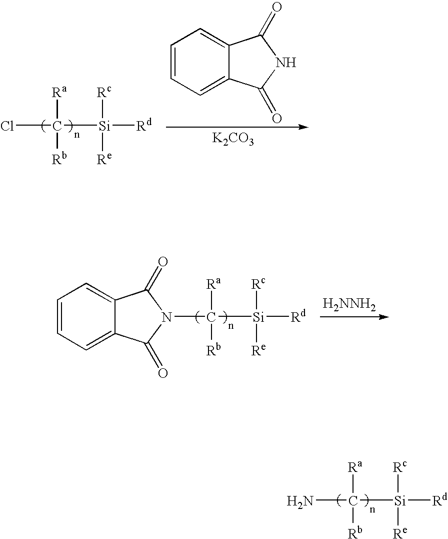

Such syntheses are described, for example, in J. Am. Chem. Soc. 1951, 73, 5130 or J. Organomet. Chem. 1978, 174, C18.

Haloalkylsilanes are commercially available or can be prepared by known methods familiar to the person skilled in the art (see, for example, Houben-Weyl, Volume 13/5, p. 65 ff. or Science of Synthesis, Vol. 4, p. 247 ff.).

The processes (a), (b) and (c) according to the invention are generally carried out under atmospheric pressure. However, it is also possible to operate under elevated or reduced pressure.

Suitable diluents for carrying out the processes (a), (b) and (c) according to the invention are all customary inert organic solvents. Preference is given to using halogenated hydrocarbons such as, for example, chlorobenzene, dichlorobenzene, dichloromethane, chloroform, carbon tetrachloride, dichloroethane or trichloroethane; ethers, such as diethyl ether, diisopropyl ether, methyl-t-butyl ether, methyl-t-amyl ether, dioxane, tetrahydrofuran, 1,2-dimethoxyethane, 1,2-diethoxyethane or anisole; nitrites, such as acetonitrile, propionitrile, n- or i-butyronitrile or benzonitrile; amides, such as N,N-dimethylfomamide, N,N-dimethylacetamide, N-methylformanilide, N-methyl-pyrrolidone or hexamethylphosphonic triamide; esters, such as methyl acetate or ethyl acetates; sulphoxides, such as dimethylsulphoxide; sulphones, such as sulpholanes.

Suitable acid acceptors for carrying out the process (a) and (b) according to the invention are all inorganic or organic base customary for such reactions. Preference is given to using alkaline earth metal or alkali metal hydrides, hydroxides, amides, alkoxides, acetates, carbonates or bicarbonates, such as, for example, sodium hydride, sodium amide, lithium diisopropylamide, sodium methoxide, sodium ethoxide, potassium tert-butoxide, sodium hydroxide, potassium hydroxide, sodium acetate, potassium acetate, calcium acetate, sodium carbonate, potassium carbonate, potassium bicarbonate and sodium bicarbonate, ad furthermore ammonium compounds, such as ammonium hydroxide, ammonium acetate and ammonium carbonate, and also tertiary amines, such as trimethylamine, triethylamine, tributylamine, N,N-dimethylaniline, N,N-dimethylbenzylamine, pyridine, N-methylpiperidine, N-methylmorpholine, N,N-dimethylaminopyridine, diazabicyclooctane (DABCO), diazabicyclononene (DBN) or diazabicycloundecene (DBU).

Suitable acid acceptors for carrying out the process (c) according to the invention are all inorganic or organic bases customary for such reactions. Preference is given to using alkali metal or alkaline earth metal acetates, carbonates or bicarbonates, such as sodium acetate, potassium acetate, calcium acetate, sodium carbonate, potassium carbonate, potassium bicarbonate or sodium bicarbonate, and furthermore ammonium compounds, such as ammonium hydroxide, ammonium acetate and ammonium carbonate, and also tertiary amines, such as trimethylamine, triethylamine, tributylamine, N,N-dimethylaniline, N,N-dimethylbenzylamine, pyridine, N-methylpiperidine, N-methylmorpholine, N,N-dimethylaminopyridine, diazabicyclooctane (DABCO), diazabicyclononene (DBN) or diazabicycloundecene (DBU).

Suitable catalysts for carrying out the process (a) and (b) and (c) according to the invention are all reaction promoters customary for such reactions. Preference is given to using fluorides, such as sodium fluoride, potassium fluoride or ammonium fluoride.

When carrying out the process (a), (b) and (c) according to the invention, the reaction temperatures can be varied within a relatively wide range. In general, the processes are carried out at temperatures between 0° C. and 150° C., preferably at temperatures between 0° C. and 80° C.

When carrying out the processes (a) and (b) according to the invention, in general from 0.5 to 10 mol, preferably from 0.8 to 2 mol, of amine of the formula (III) are employed per mole of dihalotriazolopyrimidine of the formula (II-1) and (II-2), respectively. Work-up is carried out by customary methods.

When carrying out the process (c) according to the invention, in general from 0.5 to 10 mol, preferably from 0.8 to 2 mol, of M-X¹ of the formula (IX) are employed per mole of dihalotriazolopyrimidine of the formula (II-1) and (II-2), respectively. Work-up is carried out by customary methods.

The compounds according to the invention have potent microbicidal activity and can be employed for controlling unwanted microorganisms, such as fungi and bacteria, in crop protection and in the protection of materials.

Fungicides can be employed in crop protection for controlling Plasmodiophoromycetes, Oomycetes, Chytridiomycetes, Zygomycetes, Ascomycetes, Basidiomycetes and Deuteromycetes.

Bactericides can be employed in crop protection for controlling Pseudomonadaceae, Rhizobiaceae, Enterobacteriaceae, Corynebacteriaceae and Streptomycetaceae.

Some pathogens causing fungal and bacterial diseases which come under the generic names listed above may be mentioned as examples, but not by way of limitation:
Xanthomonas species, such as, for example, Xanthomonas campestris pv. oryzae;
Pseudomonas species, such as, for example, Pseudomonas syringae pv. lachrymans;
Erwinia species, such as, for example, Erwinia amylovora;
Pythium species, such as, for example, Pythium ultimum;
Phytophthora species, such as, for example, Phytophthora infestans;
Pseudoperonospora species, such as, for example, Pseudoperonospora humuli or Pseudoperonospora cubensis;
Plasmopara species, such as, for example, Plasmopara viticola;
Bremia species, such as, for example, Bremia lactucae;
Peronospora species, such as, for example, Peronospora pisi or P. brassicae;
Erysiphe species, such as, for example, Erysiphe graminis;
Sphaerotheca species, such as, for example, Sphaerotheca fuliginea;
Podosphaera species, such as, for example, Podosphaera leucotricha;
Venturia species, such as, for example, Venturia inaequalis;
Pyrenophora species, such as, for example, Pyrenophora teres or P. graminea (conidia form: Drechslera, syn: Helminthosporium);
Cochliobolus species, such as, for example, Cochliobolus sativus (conidia form: Drechslera, syn: Helminthosporium);
Uromyces species, such as, for example, Uromyces appendiculatus;
Puccinia species, such as, for example, Puccinia recondita;
Sclerotinia species, such as, for example, Sclerotinia sclerotiorum;
Tilletia species, such as, for example, Tilletia caries;
Ustilago species, such as, for example, Ustilago nuda or Ustilago avenae;
Pellicularia species, such as, for example, Pellicularia sasaku;
Pyricularia species, such as, for example, Pyricularia oryzae;
Fusarium species, such as, for example, Fusarium culmorum;
Botrytis species, such as, for example, Botrytis cinerea;
Septoria species, such as, for example, Septoria nodorum;
Leptosphaeria species, such as, for example, Leptosphaeria nodorum;
Cercospora species, such as, for example, Cercospora canescens;
Alternaria species, such as, for example, Alternaria brassicae; and
Pseudocercosporella species, such as, for example, Pseudocercosporella herpotrichoides.

The active compounds according to the invention also show a strong invigorating action in plants. Accordingly, they are suitable for mobilizing the internal defenses of the plant against attack by unwanted microorganisms.

In the present context, plant-invigorating (resistance-inducing) compounds are to be understood as meaning substances which are capable of stimulating the defense system of plants such that, when the treated plants are subsequently inoculated with unwanted microorganisms, they display substantial resistance to these microorganisms.

In the present case, unwanted microorganisms are to be understood as meaning phytopathogenic fungi, bacteria and viruses. The compounds according to the invention can thus be used to protect plants within a certain period of time after treatment against attack by the pathogens mentioned. The period of time for which this protection is achieved is generally from 1 to 10 days, preferably 1 to 7 days, from the treatment of the plants with the active compounds.

The fact that the active compounds are well tolerated by plants at the concentrations required for controlling plant diseases permits the treatment of above-ground parts of plants, of propagation stock and seeds, and of the soil.

The active compounds according to the invention can be employed with particularly good results for controlling cereal diseases, such as, for example, against Erysiphe species, and diseases in viticulture and in the cultivation of fruit and vegetables, such as, for example, against Botrytis, Venturia, Sphaerotheca and Podosphaera species.

The active compounds according to the invention are also suitable for increasing the yield of crops. In addition, they show reduced toxicity and are well tolerated by plants.

If appropriate, the active compounds according to the invention can, at certain concentrations and application rates, also be employed as herbicides, for regulating plant growth and for controlling animal pests. If appropriate, they can also be used as intermediates or precursors in the synthesis of other active compounds.

According to the invention, it is possible to treat all plants and parts of plants. Plants are to be understood here as meaning all plants and plant populations, such as desired and undesired wild plants or crop plants (including naturally occurring crop plants). Crop plants can be plants which can be obtained by conventional breeding and optimization methods or by biotechnological and genetic engineering methods or combinations of these methods, including the transgenic plants and including plant cultivars which can or cannot be protected by plant breeders' certificates. Parts of plants are to be understood as meaning all above-ground and below-ground parts and organs of plants, such as shoot, leaf, flower and root, examples which may be mentioned being leaves, needles, stems, trunks, flowers, fruit-bodies, fruits and seeds and also roots, tubers and rhizomes. Parts of plants also include harvested material and vegetative and generative propagation material, for example seedlings, tubers, rhizomes, cuttings and seeds.

The treatment of the plants and parts of plants according to the invention with the active compounds is carried out directly or by, action on their environment, habitat or storage area according to customary treatment methods, for example by dipping, spraying, evaporating, atomizing, broadcasting, brushing-on and, in the case of propagation material, in particular in the case of seeds, furthermore by one- or multilayer coating.

In the protection of materials, the compounds according to the invention can be employed for protecting industrial materials against infection with, and destruction by, unwanted microorganisms.

Industrial materials in the present context are understood as meaning non-living materials which have been prepared for use in industry. For example, industrial materials which are intended to be protected by active compounds according to the invention from microbial change or destruction can be adhesives, sizes, paper and board, textiles, leather, wood, paints and plastic articles, cooling lubricants and other materials which can be infected with, or destroyed by, microorganisms. Parts of production plants, for example cooling-water circuits, which may be impaired by the proliferation of microorganisms may also be mentioned within the scope of the materials to be protected. Industrial materials which may be mentioned within the scope of the present invention are preferably adhesives, sizes, paper and board, leather, wood, paints, cooling lubricants and heat-transfer liquids, particularly preferably wood.

Microorganisms capable of degrading or changing the industrial materials which may be mentioned are, for example, bacteria, fungi, yeasts, algae and slime organisms. The active compounds according to the invention preferably act against fungi, in particular moulds, wood-discolouring and wood-destroying fungi (Basidiomycetes) and against slime organisms and algae.

Microorganisms of the following genera may be mentioned as examples:
Altemaria, such as Alteraria tenuis,
Aspergillus, such as Aspergillus globosum,
Chaetomium, such as Chaetomium globosum,
Coniophora, such as Coniophora puetana,
Lentinus, such as Lentinus tigrinus,
Penicillium, such as Penicillium glaucum,
Polyporus, such as Polyporus versicolor,
Aureobasidium, such as Aureobasidium pullulans,
Sclerophoma, such as Sclerophoma pityophila,
Trichoderma, such as Trichoderma viride,
Escherichia, such as Escherichia coli,
Pseudomonas, such as Pseudomonas aeruginosa, and
Staphylococcus, such as Staphylococcus aureus.

Depending on their particular physical and/or chemical properties, the active compounds can be converted into the customary formulations, such as solutions, emulsions, suspensions, powders, foams, pastes, granules, aerosols and microencapsulations in polymeric substances and in coating compositions for seeds, and ULV cool and warm fogging formulations.

These formulations are produced in a known manner, for example by mixing the active compounds with extenders, that is liquid solvents, liquefied gases under pressure, and/or solid carriers, optionally with the use of surfactants, that is emulsifiers and/or dispersants, and/or foam formers. If the extender used is water, it is also possible to employ, for example, organic solvents as auxiliary solvents. Essentially, suitable liquid solvents are: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example petroleum fractions, alcohols such as butanol or glycol and their ethers and esters, ketones such as acetone, methyl ethyl ketone, methyl isobutyl ketone or cyclohexanone, strongly polar solvents such as dimethylformamide or dimethyl sulphoxide, or else water. Liquefied gaseous extenders or carriers are to be understood as meaning liquids which are gaseous at standard temperature and under atmospheric pressure, for example aerosol propellants such as halogenated hydrocarbons, or else butane, propane, nitrogen and carbon dioxide. Suitable solid carriers are: for example ground natural minerals such as kaolins, clays, talc, chalk, quartz, attapulgite, montmorillonite or diatomaceous earth, and ground synthetic minerals such as finely divided silica, alumina and silicates. Suitable solid carriers for granules are: for example crushed and fractionated natural rocks such as calcite, pumice, marble, sepiolite and dolomite, or else synthetic granules of inorganic and organic meals, and granules of organic material such as sawdust, coconut shells, corn cobs and tobacco stalks. Suitable emulsifiers and/or foam formers are: for example nonionic and anionic emulsifiers, such as polyoxyethylene fatty acid esters polyoxyethylene fatty alcohol ethers, for example alkylaryl polyglycol ethers, alkylsulphonates, alkyl sulphates, arylsulphonates, or else protein hydrolysates. Suitable dispersants are: for example lignosulphite waste liquors and methylcellulose.

Tackifiers such as carboxymethylcellulose, natural and synthetic polymers in the form of powders, granules or lattices, such as gum arabic, polyvinyl alcohol and polyvinyl acetate, or else natural phospholipids such as cephalins and lecithins and synthetic phospholipids can be used in the formulations. Other possible additives are mineral and vegetable oils.

It is possible to use colorants such as inorganic pigments, for example iron oxide, titanium oxide and Prussian Blue, and organic dyestuffs such as alizarin dyestuffs, azo dyestuffs and metal phthalocyanine dyestuffs, and trace nutrients such as salts of iron, manganese, boron, copper, cobalt, molybdenum and zinc.

The formulations generally comprise between 0.1 and 95 percent by weight of active compound, preferably between 0.5 and 90%.

The active compounds according to the invention can, as such or in their formulations, also be used in a mixture with known fungicides, bactericides, acaricides, nematicides or insecticides, to broaden, for example, the activity spectrum or to prevent development of resistance. In many cases, synergistic effects are obtained, i.e. the activity of the mixture is greater than the activity of the individual components.

Suitable mixing components are, for example, the following compounds:

Fungicides:

2-phenylphenol; 8-hydroxyquinoline sulphate; acibenzolar-S-methyl; aldimorph; amidoflumet; ampropylfos; ampropylfos-potassium; andoprim; anilazine; azaconazole; azoxystrobin; benalaxyl; benalaxyl-M; benodanil; benomyl; benthiavalicarb-isopropyl; benzamacril; benzamacril-isobutyl; bilanafos; binapacryl; biphenyl; bitertanol; blasticidin-S; boscalid; bromuconazole; bupirimate; buthiobate; butylamine; calcium polysulphide; capsimycin; captafol; captan; carbendazim; carboxin; carpropamid; carvone; chinomethionat; chlobenthiazone;. chlorofenazole; chloroneb; chlorothalonil; chlozolinate; clozylacon; cyazofamid; cyflufenamid; cymoxanil; cyproconazole; cyprodinil; cyprofuram; Dagger G; debacarb; dichlofluanid; dichlone; dichlorophen; diclocymet; diclomezine; dicloran; diethofencarb; difenoconazole; diflumetonm; dimethirimol; dimethomorph; dimoxystrobin; diniconazole; diniconazole-M; dinocap; diphenylamine; dipyrithione; ditalimfos; dithianon; dodine; drazoxolon; edifenphos; epoxiconazole; ethaboxam; ethirimol; etridiazole; famoxadone; fenamidone; fenapanil; fenarimol; fenbuconazole; fenfuram; fenhexamid; fenitropan; fenoxanil; fenpiclonil; fenpropidin; fenpropimorph; ferbam; fluazinam; flubenzimine; fludioxonil; flumetover; flumorph; fluoromide; fluoxastrobin; fluquinconazole; fluprimidol; flusilazole; flusulfamide; flutolanil; flutriafol; folpet; fosetyl-Al; fosetyl-sodium; fuberidazole; furalaxyl; furametpyr; furcarbanil; furmecyclox; guazatine; hexachlorobenzene; hexaconazole; hymexazole; imazalil; imibenconazole; iminoctadine triacetate; iminoctadine tris(albesilate); iodocarb; ipconazole; iprobenfos; iprodione; iprovalicarb; irumamycin; isoprothiolane; isovaledione; kasugamycin; kresoxim-methyl; mancozeb; maneb; meferimzone; mepanipyrim; mepronil; metalaxyl; metalaxyl-M; metconazole; methasulfocarb; methfuroxam; metiram; metominostrobin; metsulfovax; mildiomycin; myclobutanil; myclozolin; natamycin; nicobifen; nitrothal-isopropyl; noviflumuron; nuarimol; ofurace; orysastrobin; oxadixyl; oxolinic acid; oxpoconazole; oxycarboxin; oxyfenthiin; paclobutrazole; pefurazoate; penconazole; pencycuron; phosdiphen; phihalide; picoxystrobin; piperalin; polyoxins; polyoxorim; probenazole; prochloraz; procymidone; propamocarb; propanosine-sodium; propiconazole; propineb; proquinazid; prothioconazole; pyraclostrobin; pyrazophos; pyrifenox; pyrimethanil; pyroquilon; pyroxyfur; pyrrolenitrine; quinconazole; quinoxyfen; quintozene; simeconazole; spiroxamine; sulphur; tebuconazole; tecloftalam; tecnazene; tetcyclacis; tetraconazole; thiabendazole; thicyofen; thifluzamide; thiophanate-methyl; thiram; tioxymid; tololofos-methyl; tolylfluanid; triadimefon; triadimenol; triazbutil; triazoxide; tricyclamide; tricyclazole; tridemorph; trifloxystrobin; triflumizole; triforine; triticonazole; uniconazole; validamycin A; vinclozolin; zineb; ziram; zoxamide; (2S)-N-[2-[4-[[3-(4-chlorophenyl)-2-propynyl]oxy]-3-methoxyphenyl]ethyl]-3-methyl-2-[(methylsulphonyl)amino]butanamide; 1-(1-naphthalenyl)-1H-pyrrole-2,5-dione; 2,3,5,6-tetrachloro-4-(methylsulphonyl)pyridine; 2-amino-4-methyl-N-phenyl-5-thiazolecarboxamide; 2-chloro-N-(2,3-dihydro-1,1,3-trimethyl-1H-inden-4-yl)-3-pyridinecarboxamide; 3,4,5-trichloro-2,6-pyridinedicarbonitrile; actinovate; cis-1-(4-chlorophenyl)-2-(1H-1,2,4-triazol-1-yl)cycloheptanol; methyl 1-(2,3-dihydro-2,2-dimethyl-1H-inden-1-yl)-1H-imidazole-5-carboxylate; monopotassium carbonate; N-(6-methoxy-3-pyridnyl)cyclopropanecarboxamide; N-butyl-8-(1,1-dimethylethyl)-1-oxaspiro[4.5]decane-3-amine; sodium tetracarbonate;
and copper salts and preparations, such as Bordeaux mixture; copper hydroxide; copper naphthenate; copper oxychloride; copper sulphate; cufraneb; copper oxide; mancopper; oxine-copper.
   Bactericides:
bronopol, dichlorophen, nitrapyrin, nickel dimethyldithiocarbamate, kasugamycin, octhilinone, furancarboxylic acid, oxytetracyclin, probenazole, streptomycin, tecloftalam, copper sulphate and other copper preparations.
   Insecticides/Acaricides/Nematicides:
   1. Acetylcholinesterase (AChE) Inhibitors
1.1 carbamates (for example alanycarb, aldicarb, aldoxycarb, allyxycarb, aminocarb, azamethiphos, bendiocarb, benfuracarb, bufencarb, butacarb, butocarboxim, butoxycarboxim, carbaryl, carbofuran, carbosulfan, chloethocarb, coumaphos, cyanofenphos, cyanophos, dimetilan, ethiofencarb, fenobucarb, fenothiocarb, formetanate, furathiocarb, isoprocarb, metam-sodium, methiocarb, methomyl, metolcarb, oxarmyl, pirimicarb, promecarb, propoxur, thiodicarb, thiofanox, triazamate, trimethacarb, XMC, xylylcarb)
1.2 organophosphates (for example acephate, azamethiphos, azinphos (-methyl, -ethyl), bromophos-ethyl, bromfenvinfos (-methyl), butathiofos, cadusafos, carbophenothion, chlorethoxyfos, chlorfenvinphos, chlormephos, chlorpyrifos (-methyl/-ethyl), coumaphos, cyanofenphos, cyanophos, chlorfenvinphos, demeton-S-methyl, demeton-s-methylsulphone, dialifos, diazinon, dichlofenthion, dichlorvos/DDVP, dicrotophos, dimethoate, dimethylvinphos, dioxabenzofos, disulfoton, EPN, ethion, ethoprophos, etrimfos, famphur, fenamiphos, fenitrothion, fensulfothion, fenthion, flupyrazofos, fonofos, fornothion, fosmethilan, fosthiazate, heptenophos, iodofenphos, iprobenfos, isazofos, isofenphos, isopropyl O-salicylate, isoxathion, malathion, mecarbam, methacrifos, methamidophos, methidathion, mevinphos, monocrotophos, naled, omethoate, oxydemeton-methyl, parathion (-methyl/-ethyl), phenthoate, phorate, phosalone, phosmet, phosphamidon, phosphocarb, phoxim, pirimiphos (-methyl/-ethyl), profenofos, propaphos, propetamphos, prothiofos, prothoate, pyraclofos, pyridaphenthion, pyridathion, quinalphos, sebufos, sulfotep, sulprofos, tebupirimfos, temephos, terbufos, tetrachlorvinphos, thiometon, triazophos, triclorfon, vamidothion)
   2. Sodium Channel Modulators/Blockers of Voltage-Gated Sodium Channels
2.1 pyrethroids (for example acrinathrin, allethrin (d-cis-trans, d-trans), beta-cyfluthrin, bifenthrin, bioallethrin, bioallethrin-S-cyclopentyl-isomer, bioethanomethrin, biopermethrin, bioresmethrin, chlovaporthrin, cis-cypermethrin, cis-resmethrin, cis-permethrin, clocythrin, cycloprothrin, cyfluthrin, cyhalothrin, cypermethrin (alpha-, beta-, theta-, zeta-), cyphenothrin, DDT, deltamethrin, empenthrin (1R-isomer), esfenvalerate, etofenprox, fenfluthrin, fenpropathrin, fenpyrithrin, fenvalerate, flubrocythrinate, flucythrinate, flufenprox, flumethrin, fluvalinate, fubfenprox, gamma-cyhalothrin, imiprothrin, kadethrin, lambda-cyhalothrin, metofluthrin, permethrin (cis-, trans-), phenothrin (1R-trans isomer), prallethrin, profluthrin, protrifenbute, pyresmethrin, resmethrin, RU 15525, silafluofen, tau-fluvalinate, teflutlrin, terallethrin, tetramethrin-(1R-isomer), tralomethrin, transfluthrin, ZXI 8901, pyrethrins (pyrethrum))
2.2 oxadazines (for example indoxacarb)
   3. Acetylcholine Receptor Agonists/Antagonists
3.1 chloronicotinyls/neonicotinoids (for example acetampiprid, clothianidin, dinotefuran, imidacloprid, nitenpyram, nithiazine, thiacloprid, thiamethoxam)
3.2 nicotine, bensultap, cartap
   4. Acetylcholine Receptor Modulators
4.1 spinosyns (for example spinosad)
   5. Antagonists of GABA-Gated Chloride Channels
5.1 cyclodiene organochlorines (for example camphechlor, chlordane, endosulphan, gamma-HCH, HCH, heptachlor, lindane, methoxychlor
5.2 fiproles (for example acetoprole, ethiprole, fipronil, vaniliprole)
   6 Chloride Channel Activators
6.1 mectins (for example abamectin, avermectin, emamectin, emamectin-benzoate, ivermectin, milbemectin, milbemycin)
   7. Juvenile Hormone Mimetics (for example diofenolan, epofenonane, fenoxycarb, hydroprene, kinoprene, methoprene, pyriproxifen, triprene)
   8. Ecdyson Agonists/Disruptors
8.1 diacylhydrazines (for example chromafenozide, halofenozide, methoxyfenozide, tebufenozide)
   9. Chitin Biosynthesis Inhibitors
9.1 benzoylureas (for example bistrifluron, chlofluazuron, diflubenzuron, fluazuron, flucycloxuron, flufenoxuron, hexaflumuron, lufenuron, novaluron, noviflumuron, penfluron, teflubenzuron, triflumuron)
9.2 buprofezin
9.3 cyromazine
   10 Inhibitors of Oxidative Phosphorylation, ATP Disruptors
10.1 diafenthiuron
10.2 organotins (for example azocyclotin, cyhexatin, fenbutatin-oxide)
   11. Decouplers of Oxidativephosphorylation Acting by Interrupting the H-Proton Gradient
11.1 pyrroles (for example chlorfenapyr)
11.2 dinitrophenols (for example binapacryl, dinobuton, dinocap, DNOC)
   12. Site-I Electron Transport Inhibitors
12.1 METIs (for example fenazaquin, fenpyroximate, pyrimidifen, pyridaben, tebufenpyrad, tolfenpyrad)
12.2 hydramethylnone
12.3 dicofol
   13. Site-II Electron Transport Inhibitors
13.1 rotenone
   14. Site-III Electron Transport Inhibitors
14.1 acequinocyl, fluacrypyrim
   15. Microbial Disruptors of the Insect Gut Membrane
Bacillus thuringiensis strains
   16. Inhibitors of Fat Synthesis
16.1 tetronic acids (for exampie spirodiclofen, spiromesifen)
16.2 tetramic acids [for example 3-(2,5-dimethylphenyl)-8-methoxy-2-oxo-1-azaspiro[4.5]dec-3-en-4-yl ethyl carbonate (alias: carbonic acid, 3-(2,5-dimethylphenyl)-8-methoxy-2-oxo-1-azaspiro[4.5]dec-3-en-4-yl ethyl ester, CAS Reg. No.: 382608-10-8) and carbonic acid, cis-3-(2,5dimethylphenyl)-8-methoxy-2-oxo-1-azaspiro[4.5]dec-3-en-4-yl ethyl ester (CAS Reg. No.: 203313-25-1)]
   17. Carboxamides
(for example flonicamid)
   18. Octopaminergic Agonists
(for example amitraz)
   19. Inhibitors of Magnesium-Stimulated ATPase
(for example propargite)
   20. Phthalamides
(for example N²-[1,1-dimethyl-2-(methylsulphonyl)ethyl]-3-iodo-N¹-[2-methyl-4-[1,2,2,2-tetrafluoro-1-(trifluoromethyl)ethyl]phenyl]-1,2-benzenedicarboxamide (CAS Reg. No.: 272451-65-7), flubendiamide)
   21. Nereistoxin Analogues
(for example thiocyclam hydrogen oxalate, thiosultap-sodium)
   22. Biologicals, Hormones or Pheromones
(for example azadirachtin, Bacillus spec., Beauveria spec., codlemone, Metarrhizium spec., Paecilomyces spec., thuringiensin, Verticillium spec.)
   23. Active Compounds with Unknown or Unspecific Mechanisms of Action
23.1 fumigants (for example aluminium phosphide, methyl bromide, sulphuryl fluoride)
23.2 selective antifeedants (for example cryolite, flonicamid, pymetrozine)
23.3 mite growth inhibitors (for example clofentezine, etoxazole, hexythiazox)
23.4 amidoflumet, benclothiaz, benzoxumate, bifenazate, bromopropylate, buprofezin, chinomethionat, chlordimeform, chlorobezzilate, chloropicrin, clothiazoben,cycloprene, cyflumetofen, dicyclanil, fenoxacrim, fentrifanil, flubenzinmine, flufenerim, flutenzin, gossyplure, hydramethylnone, japonilure, metoxadiazone, petroleum, piperonyl butoxide, potassium oleate, pyrafluprole, pyridalyl, pyriprole, sulfluramid, tetradifon, tetrasul, triarathene, verbutin,
furthermore the compound 3-methylphenyl propylcarbamate (Tsumacide Z), the compound 3-(5-chloro-3-pyridinyl)-8-(2,2,2-trifluoroethyl)-8-azabicyclo[3.2.1]octane-3-carbonitrile (CAS Reg. No. 185982-80-3) and the corresponding 3-endo-isomer (CAS Reg. No. :185984-60-5) (cf. WO 96/37494, WO 98/25923), and preparations which comprise insecticidally active plant extracts, nematodes, fungi or viruses.

A mixture with other known active compounds, such as herbicides, or with fertilizers and growth regulators, safeners and/or semiochemicals is also possible.

In addition, the compounds of the formula (I) according to the invention also have very good antimycotic activity. They have a very broad antimycotic activity spectrum in particular against dermatophytes and yeasts, moulds and diphasic fungi (for example against Candida species such as Candida albicans, Candida glabrata) and Epidermophyton floccosum, Aspergillus species such as Aspergillus niger and Aspergillus fumigatus, Trichophyton species such as Trichophyton mentagrophytes, Microsporon species such as Microsporon canis and audouinii. The list of these fungi does by no means limit the mycotic spectrum which can be covered, but is only for illustration.

The active compounds can be used as such, in the form of their formulations or the use forms prepared therefrom, such as ready-to-use solutions, suspensions, wettable powders, pastes, soluble powders, dusts and granules. Application is carried out in a customary manner, for example by watering, spraying, atomizing, broadcasting, dusting, foaming, spreading, etc. It is furthermore possible to apply the active compounds by the ultra-low volume method, or to inject the active compound preparation or the active compound itself into the soil. It is also possible to treat the seeds of the plants.

When using the active compounds according to the invention as fungicides, the application rates can be varied within a relatively wide range, depending on the kind of application. For the treatment of parts of plants, the active compound application rates are generally between 0.1 and 10 000 g/ha, preferably between 10 and 1000 g/ha. For seed dressing, the active compound application rates are generally between 0.001 and 50 g per kilogram of seed, preferably between 0.01 and 10 g per kilogram of seed. For the treatment of the. soil, the active compound application rates are generally between 0.1 and 10 000 g/ha, preferably between 1 and 5 000 g/ha.

As already mentioned above, it is possible to treat all plants and their parts according to the invention. In a preferred embodiment, wild plant species and plant cultivars, or those obtained by conventional biological breeding, such as crossing or protoplast fusion, and parts thereof, are treated. In a further preferred embodiment, transgenic plants and plant cultivars obtained by genetic engineering, if appropriate in combination with conventional methods (Genetically Modified Organisms), and parts thereof, are treated. The term “parts” or “parts of plants” or “plant parts” has been explained above.

Particularly preferably, plants of the plant cultivars which are in each case commercially available or in use are treated according to the invention. Plant cultivars are to be understood as meaning plants having new properties (“traits”) and which have been obtained by conventional breeding, by mutagenesis or by recombinant DNA techniques. They can be cultivars, varieties, bio- or genotypes.

Depending on the plant species or plant cultivars, their location and growth conditions (soils, climate, vegetation period, diet), the treatment according to the invention may also result in superadditive (“synergistic”) effects. Thus, for example, reduced application rates and/or a widening of the activity spectrum and/or an increase in the activity of the substances and compositions which can be used according to the. invention, better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products are possible which exceed the effects which were actually to be expected.

The transgenic plants or plant cultivars (i.e. those obtained by genetic engineering) which are preferably to be treated according to the invention include all plants which, in the genetic modification, received genetic material which imparted particularly advantageous useful properties (“traits”) to these plants. Examples of such properties are better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products. Further and particularly emphasized examples of such properties are a better defence of the plants against animal. and microbial pests, such as against insects, mites, phytopathogenic fungi, bacteria and/or viruses, and also increased tolerance of the plants to certain herbicidally active compounds. Examples of transgenic plants which may be mentioned are the important crop plants, such as cereals (wheat, rice), corn,soybeans, potatoes, cotton, tobacco, oilseed rape and also fruit plants (with the fruits apples, pears, citrus fruits and grapes), and particular emphasis is given to corn, soybeans, potatoes, cotton, tobacco and oilseed rape. Traits that are particularly emphasized are increased defence of the plants against insects, arachnids,nematodes and slugs and snails by toxins formed in the plants, in particular those formed in the plants by the genetic material from Bacillus thuringiensis (for example by the genes CryIA(a), CryIA(b), CryIA(c), CryIIA, CryIIIA, CryIIIB2, Cry9c, Cry2Ab, Cry3Bb and CryIF and also combinations thereof) (hereinbelow referred to as “Bt plants”). Traits that are also particularly emphasized are the increased defence of the plants against fungi, bacteria and viruses by systemic acquired resistance (SAR), systemin, phytoalexins, elicitors and resistance genes and correspondingly expressed proteins and toxins. Traits that are furthermore particularly emphasized are the increased tolerance of the plants to certain herbicidally active compounds, for example imidazolinones, sulphonylureas, glyphosate or phosphinotricin (for example the “PAT” gene). The genes which impart the desired traits in question can also be present in combination with one another in the transgenic plants. Examples of “Bt plants” which may be mentioned are corn varieties, cotton varieties, soybean varieties and potato varieties which are sold under the trade names YIELD GARD® (for example corn, cotton, soybeans), KnockOut® (for example corn), StarLink® (for example corn), Bollgard® (cotton), Nucoton® (cotton) and NewLeaf® (potato). Examples of herbicide-tolerant plants which may be mentioned are corn varieties, cotton varieties and soybean varieties which are sold under the trade names Roundup Ready® (tolerance to glyphosate, for example corn, cotton, soybean), Liberty Link® (tolerance to phosphinotricin, for example oilseed rape), IMI® (tolerance to imidazolinones) and STS® (tolerance to sulphonylureas, for example corn). Herbicide-resistant plants (plants bred in a conventional manner for herbicide tolerance) which may be mentioned also include the varieties sold under the name Clearfield® (for example corn). Of course, these statements also apply to plant cultivars which have these genetic traits or genetic traits still to be developed, and which will be developed and/or marketed in the future.

The plants listed can be treated according to the invention in a particularly advantageous manner with the compounds of the general formula (I) or the active compound mixtures according to the invention. The preferred ranges stated above for the active compounds or nmixtures also apply to the treatment of these plants. Particular emphasis is given to the treatment of plants with the compounds or mixtures specifically mentioned in the present text.

The compounds of the formula (I) according to the invention are furthermore suitable for suppressing the growth of tumour cells in humans and mammals. This is based on an interaction of the compounds according to the invention with tubulin and microtubuli and by promoting microtubuli polymerization.

For this purpose, it is possible to administer an effective amount of one or more compounds of the formula (I) or pharmaceutically acceptable salts thereof.

The preparation and the use of the active compounds according to the invention are illustrated in the examples below.

### EXAMPLES

### Example 1

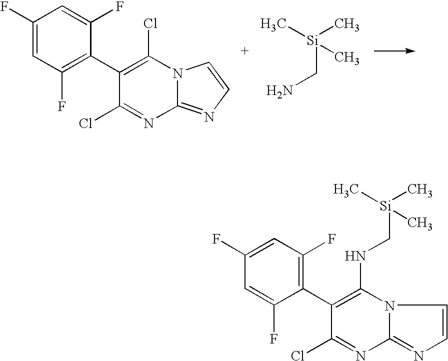

0.5 g (0.002 mol) of 6-(2,4,6-trifluorophenyl)-5,7-dichloroimidazo[1,2-a]pyrimidine was initially charged in 7.8 g of acetonitrile. At room temperature, 0.326 g (0.002 mol) of potassium carbonate and 0.162 g (0.002 mol) of trimethylsilylmethylamine were added and the mixture was stirred for 16 hours. The reaction mixture was acidified with hydrochloric acid and extracted with diethyl ether. The organic phase was dried and concentrated. The residue was stirred with diethyl ether, filtered off with suction and dried. This gave 0.2 g of 5-chloro-6-(2,4,6-trifluorophenyl-)-7-trimethylsilylmethylaminoimidazo[1,2-a]pyrimidine (log p=2.09; content according to HPLC 98.6%)

### Example 2

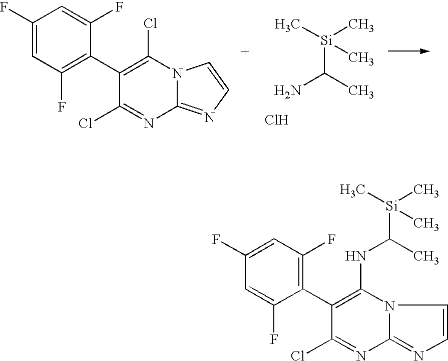

0.5 g (0.002 mol) of 6-(2,4,6-trifluorophenyl)-5,7-dichloroimidazo[1,2-a]pyrimidine was initially charged in 7.8 g of acetonitrile. At room temperature, 0.543 g (0.004 mol) of potassium carbonate and 0.242 g (0.002 mol) of 2-trimethylsilyl-1-aminoethane were added and the mixture was stirred for 16 hours. The reaction mixture was acidified with hydrochloric acid and extracted with diethyl ether. The organic phase was dried and concentrated and then, in cyclohexane:ethyl acetate=1:1, chromatographed on silica gel. This gave 0.3 g of 5-chloro-6-(2,4,6-trifloropheny)-7-(1-trimethylsilylethylamino)imidazo[1,2-a]pyrimidine (log p=2.18; content according to HPLC 87.4%)

The compound 6-(2,4,6-trifluorophenyl)-5,7-dichloroimidazo[1,2-a]pyrimidine is known from WO-A 03/022 850 and from WO-A 03/089 433.

The compounds of the formula (I-a) listed in Table 1 below are or were obtained analogously to the methods given above

TABLE 1

| | |
|---|---|
| | (I-a) |
| 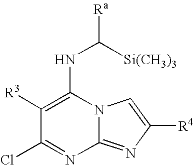 | |

| Ex. | | | | | |
|---|---|---|---|---|---|
| No. | R^{a} | R³ | R⁴ | logP | Mp. (° C.) |
| 1 | H | 2,4,6-trifluorophenyl | H | 2.09 | |
| 2 | CH₃ | 2,4,6-trifluorophenyl | H | 2.18 | |
| 3 | H | 2-Cl-4-F-phenyl | H | 2.20 | |
| 4 | CH₃ | 2-Cl-4-F-phenyl | H | 2.38 | |
| 5 | H | 2-Cl-6-F-phenyl | H | | |
| 6 | CH₃ | 2-Cl-6-F-phenyl | H | | |
| 7 | CH₃ | 2-Cl-phenyl | H | | |
| 8 | H | 3-Cl-5-(CF₃)-pyridin-2-yl | H | | |
| 9 | CH₃ | 5-F-pyrimidin-4-yl | H | | |
| 10 | CH₃ | 3-(CF₃)-pyridin-2-yl | H | | |
| 11 | CH₃ | 2-Cl-6-F-phenyl | H | | |
| 12 | H | 2-Cl-6-F-phenyl | H | | |
| 13 | H | 2,5-difluorophenyl | H | | |
| 14 | CH₃ | 2,5-difluorophenyl | H | | |
| 15 | CH₃ | 2,5-difluorophenyl | H | | |
| 16 | H | 2,5-difluorophenyl | H | | |
| 17 | CH₃ | 2,5-difluorophenyl | H | | |
| 18 | H | 2,5-difluorophenyl | H | | |
| 19 | CH₃ | 5-F-pyrimidin-4-yl | H | | |
| 20 | H | 2-Cl-phenyl | H | | |
| 21 | CH₃ | 2-Cl-phenyl | H | | |
| 22 | H | 5-F-pyrimidin-4-yl | H | | |
| 23 | CH₃ | 5-Cl-pyrimidin-4-yl | H | | |
| 24 | H | 5-Cl-pyrimidin-4-yl | H | | |
| 25 | CH₃ | sec-butyl | H | | |
| 26 | H | sec-butyl | H | | |
| 27 | H | 5-F-pyrimidin-4-yl | H | | |
| 28 | CH₃ | 5-F-pyrimidin-4-yl | H | | |
| 29 | H | N(—CH₂—CH₂—CH₂—CH₂—CH₂—) | H | | |
| 30 | CH₃ | N(—CH₂—CH₂—CH₂—CH₂—CH₂—) | H | | |
| 31 | H | N(—CHCH₃—CH₂—CH₂—CH₂—CH₂—) | H | | |
| 32 | H | N(—CHCH₃—CH₂—CH₂—CH₂—CH₂—) | H | | |
| 33 | H | N(—CHCH₃—CH₂—O—CH₂—CH₂—) | H | | |
| 34 | CH₃ | N(—CHCH₃—CH₂—O—CH₂—CH₂—) | H | | |
| 35 | H | 3,5-dimethylpyrazol-1-yl | H | | |
| 36 | CH₃ | 3,5-dimethylpyrazol-1-yl | H | | |
| 37 | H | N(CH₃)(C₂H₅) | H | | |
| 38 | CH₃ | N(CH₃)(C₂H₅) | H | | |
| 39 | H | 3-thienyl | H | | |
| 40 | CH₃ | 3-thienyl | H | | |
| 41 | H | 3-F-phenylthio | H | | |
| 42 | CH₃ | 3-F-phenylthio | H | | |
| 43 | H | 3-Cl-phenylthio | H | | |
| 44 | CH₃ | 3-Cl-phenylthio | H | | |
| 45 | H | 3-F-phenyl-CH₂ | H | | |
| 46 | CH₃ | 3-F-phenyl-CH₂ | H | | |
| 47 | H | 3-Cl-phenyl-CH₂ | H | | |
| 48 | CH₃ | 3-Cl-phenyl-CH₂ | H | | |

The logP values were determined in accordance with EEC Directive 79/831 Annex V. A8 by HPLC (gradient method, acetonitrile/0.1% aqueous phosphoric acid).

The compounds of the formula (I-b) listed in Table 2 below are or were likewise obtained analogously to the methods given above.

TABLE 2

| | |
|---|---|
| | (I-b) |
| 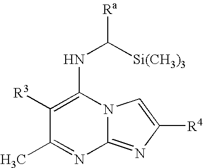 | |

| Ex. | | | | | |
|---|---|---|---|---|---|
| No. | R^{a} | R³ | R⁴ | logP | Mp. (° C.) |
| 1 | H | 2,4,6-trifluorophenyl | H | | |
| 2 | CH₃ | 2,4,6-trifluorophenyl | H | | |
| 3 | H | 2-Cl-4-F-phenyl | H | | |
| 4 | CH₃ | 2-Cl-4-F-phenyl | H | | |
| 5 | H | 2-Cl-6-F-phenyl | H | | |
| 6 | CH₃ | 2-Cl-6-F-phenyl | H | | |
| 7 | CH₃ | 2-Cl-phenyl | H | | |
| 8 | H | 3-Cl-5-(CF₃)-pyridin-2-yl | H | | |
| 9 | CH₃ | 5-F-pyrimidin-4-yl | H | | |
| 10 | CH₃ | 3-(CF₃)-pyridin-2-yl | H | | |
| 11 | CH₃ | 2-Cl-6-F-phenyl | H | | |
| 12 | H | 2-Cl-6-F-phenyl | H | | |
| 13 | H | 2,5-difluorophenyl | H | | |
| 14 | CH₃ | 2,5-difluorophenyl | H | | |
| 15 | CH₃ | 2,5-difluorophenyl | H | | |
| 16 | H | 2,5-difluorophenyl | H | | |
| 17 | CH₃ | 2,5-difluorophenyl | H | | |
| 18 | H | 2,5-difluorophenyl | H | | |
| 19 | CH₃ | 5-F-pyrimidin-4-yl | H | | |
| 20 | H | 2-Cl-phenyl | H | | |
| 21 | CH₃ | 2-Cl-phenyl | H | | |
| 22 | H | 5-F-pyrimidin-4-yl | H | | |
| 23 | CH₃ | 5-Cl-pyrimidin-4-yl | H | | |
| 24 | H | 5-Cl-pyrimidin-4-yl | H | | |
| 25 | CH₃ | sec-butyl | H | | |
| 26 | H | sec-butyl | H | | |
| 27 | H | 5-F-pyrimidin-4-yl | H | | |
| 28 | CH₃ | 5-F-pyrimidin-4-yl | H | | |
| 29 | H | N(—CH₂—CH₂—CH₂—CH₂—CH₂—) | H | | |
| 30 | CH₃ | N(—CH₂—CH₂—CH₂—CH₂—CH₂—) | H | | |
| 31 | H | N(—CHCH₃—CH₂—CH₂—CH₂—CH₂—) | H | | |
| 32 | H | N(—CHCH₃—CH₂—CH₂—CH₂—CH₂—) | H | | |
| 33 | H | N(—CHCH₃—CH₂—O—CH₂—CH₂—) | H | | |
| 34 | CH₃ | N(—CHCH₃—CH₂—O—CH₂—CH₂—) | H | | |
| 35 | H | 3,5-dimethylpyrazol-1-yl | H | | |
| 36 | CH₃ | 3,5-dimethylpyrazol-1-yl | H | | |
| 37 | H | N(CH₃)(C₂H₅) | H | | |
| 38 | CH₃ | N(CH₃)(C₂H₅) | H | | |
| 39 | H | 3-thienyl | H | | |
| 40 | CH₃ | 3-thienyl | H | | |
| 41 | H | 3-F-phenylthio | H | | |
| 42 | CH₃ | 3-F-phenylthio | H | | |
| 43 | H | 3-Cl-phenylthio | H | | |
| 44 | CH₃ | 3-Cl-phenylthio | H | | |
| 45 | H | 3-F-phenyl-CH₂ | H | | |
| 46 | CH₃ | 3-F-phenyl-CH₂ | H | | |
| 47 | H | 3-Cl-phenyl-CH₂ | H | | |
| 48 | CH₃ | 3-Cl-phenyl-CH₂ | H | | |

The logP values were determined in accordance with EEC Directive 79/831 Annex V. A8 by HPLC (gradient method, acetonitrile/0.1% aqueous phosphoric acid).

### USE EXAMPLES

### Example A

In Vitro Test for Determining the ED₅₀ of Microorganisms

A methanolic solution of the active compound to be tested, mixed with the emulsifier PS16, is pipetted into the cavities of microtiter plates. After evaporation of the solvent, 200 μl of potato dextrose medium are added to each cavity.

Beforehand, a suitable concentration of spores or mycelium of the fungus to be tested was added to the medium.

The resulting concentrations of the active compound are 0.05, 0.5, 5 and 50 ppm. The resulting concentration of the emulsifier is 300 ppm.

The plates are then incubated on a shaker at a temperature of 200° C. for 3-5 days until sufficient growth can be detected in the untreated control.

Evaluation is carried out photometrically at a wavelength of 620 nm. The dose of active compound resulting in 50% inhibition of the fungal growth compared to the untreated control (ED₅₀) is calculated from the data measured for different concentrations.

Here, the compound of Example 2 showed an ED₅₀ value of smaller than <1 for Altemaria mali, Botrytis cinerea and Ustilago avenae.

### Example B

Botrytis test (cucumber)/protective
Solvent: 49 parts by weight of N,N-dimethylformamide
Emulsifier: 1 part by weight of alkylaryl polyglycol ether

To produce a suitable preparation of active compound, 1 part by weight of active compound is mixed with the stated amounts of solvent and emulsifier, and the concentrate is diluted with water to the desired concentration.

To test for protective activity, young cucumber plants are sprayed with the preparation of active compound at the stated application rate. One day aft er the treatment, the plants are inoculated with a spore suspension of Botrytis cinerea and then remain at 100% relative hunmdity and 20° C. for 48 h. The plants then remain at 96% relative atmospheric-humidity and a temperature of 10° C.

Evaluation is carried out 5-6 days after the inoculation. 0% means an efficacy which corresponds to that of the control, whereas an efficacy of 100% means that no infection is observed.

Here, the compound of Example 2 showed, at an application rate of 750 g/ha, an efficacy of >80%.

What is claimed is:

1-10. (canceled)

11. An imidazolopyrimidine of formula (I) 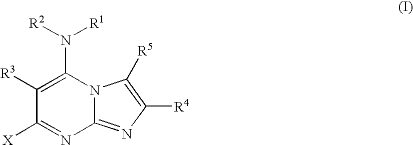
and salts thereof,
in which
R¹ represents H, optionally substituted alkyl, optionally substituted alkenyl, optionally substituted alkynyl, optionally substituted cycloalkyl, or optionally substituted heterocyclyl; or represents an organic radical that contains 3 to 13 carbon atoms and one or more silicon atoms and optionally 1 to 3 identical or different heteroatoms selected from the group consisting of oxygen, nitrogen, and sulphur and which that is unsubstituted or substituted by 1 to 4 identical or different halogens,
R² represents an organic radical that contains 3 to 13 carbon atoms and one or more silicon atoms and optionally 1 to 3 identical or different heteroatoms selected from the group consisting of oxygen, nitrogen, and sulphur and which that is unsubstituted or substituted by 1 to 4 identical or different halogens, or
R¹ and R² together with the nitrogen atom to which they are attached represent an optionally substituted heterocyclic ring that contains one or more silicon atoms and/or is substituted by one or more radials R²,
R³ represents optionally substituted aryl, optionally substituted heterocyclyl, optionally substituted alkyl, optionally substituted alkenyl, optionally substituted alkynyl, optionally substituted cycloalkyl, optionally substituted aralkyl, halogen, or optionally substituted amino, optionally substituted (C₁-C₈)-alkoxy, optionally substituted (C₁-C₈)-alkylthio, optionally substituted (C₆-C₁₀)-aryloxy, optionally substituted (C₆-C₁₀)-arylthio, optionally substituted heterocyclyloxy, optionally substituted (C₆-C₁₀)-aryl-(C₁-C₄)-alkoxy, optionally substituted (C₆-C₁₀)-aryl-(C₁-C₄)-alkylthio, optionally substituted heterocyclyl-(C₁-C₄)-alkoxy, or optionally substituted heterocyclyl-(C₁-C₄)-alkylthio,
R⁴ represents H, halogen, optionally halogen-substituted alkyl, or optionally halogen-substituted cycloalkyl,
R⁵ represents H, halogen, optionally halogen-substituted alkyl, or optionally halogen-substituted cycloalkyl, and
X represents halogen, cyano, optionally substituted alkyl, optionally substituted alkoxy, or optionally substituted phenyl.

12. An imidazolopyrimidine of formula (I) according to claim 11 in which
R¹ represents H; represents alkyl having 1 to 6 carbon atoms that is optionally mono- to pentasubstituted by identical or different substituents selected from the group consisting of halogen, cyano, hydroxy, alkoxy having 1 to 4 carbon atoms, and cycloalkyl having 3 to 8 carbon atoms; represents alkenyl having 2 to 6 carbon atoms that is optionally mono- to trisubstituted by identical or different substituents selected from the group consisting of halogen, cyano, hydroxy, alkoxy having 1 to 4 carbon atoms, and cycloalkyl having 3 to 8 carbon atoms; represents alkynyl having 3 to 6 carbon atoms that is optionally mono- to trisubstituted by identical or different substituents selected from the group consisting of halogen, cyano, alkoxy having 1 to 4 carbon atoms, and cycloalkyl having 3 to 8 carbon atoms; represents cycloalkyl having 3 to 8 carbon atoms that is optionally mono- to trisubstituted by identical or different substituents selected from the group consisting of halogen and alkyl having 1 to 4 carbon atoms; represents saturated or unsaturated heterocyclyl having 3 to 8 ring members and 1 to 3 heteroatoms selected from the group consisting of nitrogen, oxygen, and sulphur, where the heterocyclyl is optionally mono- or disubstituted by halogen, alkyl having 1 to 4 carbon atoms, cyano, and/or cycloalkyl having 3 to 8 carbon atoms; or represents an aliphatic saturated or unsaturated group having 1 to 13 carbon atoms and one or more silicon atoms that optionally contains 1 to 3 identical or different heteroatoms selected from the group consisting of oxygen, sulphur, and nitrogen and that is unsubstituted or substituted by 1 to 4 identical or different halogen atoms,
R² represents an aliphatic saturated or unsaturated group having 1 to 13 carbon atoms and one or more silicon atoms that optionally contains 1 to 3 identical or different heteroatoms selected from the group consisting of oxygen, sulphur, and nitrogen and that is unsubstituted or substituted by 1 to 4 identical or different halogen atoms, or
R¹ and R² together with the nitrogen atom to which they are attached represent a saturated or unsaturated heterocyclic ring having 3 to 8 ring members that contains one or more silicon atoms and/or is substituted by one or more radicals R², where the heterocycle optionally contains a further nitrogen, oxygen or sulphur atom as ring member and where the heterocycle is optionally substituted up to three times by fluorine, chlorine, bromine, alkyl having 1 to 4 carbon atoms, and/or haloalkyl having 1 to 4 carbon atoms and 1 to 9 fluorine and/or chlorine atoms;
R³ represents C₁-C₁₀-alkyl, C₂-C₁₀-alkenyl, C₂-C₁₀-alkynyl, C₃-C₈-cycloalkyl, or phenyl-C₁-C₁₀-alkyl, where each such group is unsubstituted or partially or fully halogenated and/or optionally carries one to three radicals R^{X}; represents C₁-C₁₀-haloalkyl that optionally carries one to three radicals R^{X}; represents phenyl that is optionally mono- to tetrasubstituted by identical or different substituents selected from the group consisting of halogen, cyano, nitro, amino, hydroxy, formyl, carboxy, carbamoyl, and thiocarbamoyl; of straight-chain or branched alkyl, alkoxy, alkylthio, alkylsulphinyl, and alkylsulphonyl having in each case 1 to 6 carbon atoms, of straight-chain or branched alkenyl and alkenyloxy having in each case 2 to 6 carbon atoms, of straight-chain or branched haloalkyl, haloalkoxy, haloalkylthio, haloalkylsulphinyl, and haloalkylsulphonyl having in each case 1 to 6 carbon atoms and 1 to 13 identical or different halogen atoms, of straight-chain or branched haloalkenyl and haloalkenyloxy having in each case 2 to 6 carbon atoms and 1 to 11 identical or different halogen atoms, of straight-chain or branched alkylamino, dialkylamino, alkylcarbonyl, alkylcarbonyloxy, alkoxycarbonyl, alkylsulphonyloxy, hydroximinoalkyl, and alkoximinoalkyl having in each case 1 to 6 carbon atoms in the individual alkyl moieties, of cycloalkyl having 3 to 8 carbon atoms, and of 1,3-propanediyl, 1,4-butanediyl, methylenedioxy (—O—CH₂—O—), and 1,2-ethylenedioxy (—O—CH₂—CH₂—O—), each of which is attached in the 2,3-position and is optionally mono- or polysubstituted by identical or different substituents selected from the group consisting of halogen, alkyl having 1 to 4 carbon atoms, and haloalkyl having 1 to 4 carbon atoms and 1 to 9 identical or different halogen atoms; represents saturated or unsaturated heterocyclyl having 3 to 8 ring members and 1 to 3 heteroatoms selected from the group consisting of nitrogen, oxygen, and sulphur, where the heterocyclyl is optionally mono- or disubstituted by halogen, alkyl having 1 to 4 carbon atoms, alkoxy having 1 to 4 carbon atoms, alkylthio having 1 to 4 carbon atoms, haloalkoxy having 1 to 4 carbon atoms, haloalkylthio having 1 to 4 carbon atoms, cyano, nitro, and/or cycloalkyl having 3 to 6 carbon atoms; or represents C₁-C₈-alkylamino, C₂-C₈-alkenylamino, C₂-C₈-alkynylamino, di-C₁-C₈-alkylamino, di-C₂-C₈-alkenylamino, di-C₂-C₈-alkynylamino, C₂-C₈-alkenyl-(C₂-C₈)-alkynylamino, C₂-C₆-alkynyl-(C₁-C₈)-alkylamino, C₂-C₈-alkenyl-(C₁-C₈)-alkylamino, C₆-C₁₀-arylamino, C₆-C₁₀-aryl-(C₁-C₈)-alkyl-amino, C₆-C₁₀-aryl-(C₁-C₄)-alkyl-(C₁-C₈)-alkylamino, heterocyclyl-(C₁-C₈)-alkylamino, or heterocyclyl-(C₁-C₄)-alkyl-(C₁-C₈)-alkylamino; where R^{X} represents cyano, nitro, hydroxy, C₁-C₆-alkyl, C₁-C₆-haloalkyl, C₃-C₆-cycloalkyl, C₁-C₆-alkoxy, C₁-C₆-haloalkoxy, C₁-C₆-alkylthio, C₁-C₆-haloalkylthio, C₁-C₆-alkylsulphinyl, C₁-C₆-haloalkylsulphinyl, C₁-C₆-alkyl-sulphonyl, C₁-C₆-haloalkylsulphonyl, C₁-C₆-alkylamino, di-C₁-C₆-alkylamino, C₂-C₆-alkenyl, C₂-C₆-alkenyloxy, C₂-C₆-alkynyl, or C₃-C₆-alkynyloxy; or represents optionally halogenated oxy-C₁-C₄-alkyl-C₁-C₄-alkenoxy, oxy-C₁-C₄-alkenyl-C₁-C₄-alkoxy, or oxy-C₁-C₄-alkyl-C₁-C₄-alkyloxy,
R⁴ represents H, halogen, (C₁-C₄)-alkyl that is unsubstituted or substituted by one or more halogen atoms, or cyclopropyl that is unsubstituted or substituted by one or more halogen atoms,
R⁵ represents H, halogen, (C₁-C₄)-alkyl that is unsubstituted or substituted by one or more halogen atoms, or cyclopropyl that is unsubstituted or substituted by one or more halogen atoms, and
X represents H, fluorine, chlorine, bromine, or CN.

13. An imidazolopyrimidine of formula (I) according to claim 11 in which
R¹ represents hydrogen, methyl, or ethyl;
R² represents a group Y²—Si(OₘCH₃)(OₙCH₃)(OₚY³),
where
m, n, and p independently of one another represent 0 or 1,
Y² represents a bond or alkanediyl, alkenediyl, or alkynediyl that are in each case straight-chain or branched, have 1 to 6 and 2 to 6 carbon atoms, are optionally interrupted by one or two non-adjacent oxygen atoms, and are unsubstituted or substituted by 1 to 3 identical or different halogen atoms, and
Y³ represents straight-chain or branched alkyl or alkenyl that have 1 to 5 and 2 to 5 carbon atoms, are optionally interrupted by an oxygen, nitrogen, or sulphur atom, and are unsubstituted or substituted by 1 to 3 identical or different halogen atoms;
R³ represents (C₁-C₈)-alkyl, (C₁-C₈)-cycloalkyl, or benzyl; represents phenyl that is optionally mono- to trisubstituted by identical or different substituents selected from the group consisting of fluorine, chlorine, bromine, cyano, nitro, formyl, methyl, ethyl, n- or i-propyl, n-, i-, s-, or t-butyl, allyl, propargyl, methoxy, ethoxy, n- or i-propoxy, methylthio, ethylthio, n- or i-propylthio, methylsulphinyl, ethylsulphinyl, methylsulphonyl, ethylsulphonyl, allyloxy, propargyloxy, trifluoromethyl, trifluoroethyl, difluoromethoxy, trifluoromethoxy, difluorochloromethoxy, trifluoroethoxy, difluoromethylthio, difluorochloromethylthio, trifluoromethylthio, trifluoromethylsulphinyl, trifluoromethylsulphonyl, trichloroethynyloxy, trifluoroethynyloxy, chloroallyloxy, iodopropargyloxy, methylamino, ethylamino, n- or i-propylamino, dimethylamino, diethylamino, acetyl, propionyl, acetyloxy, methoxycarbonyl, ethoxycarbonyl, hydroximinomethyl, hydroximinoethyl, methoximinomethyl, ethoximinomethyl, methoximinoethyl, ethoximinoethyl, cyclopropyl, cyclobutyl, cyclopentyl, and cyclohexyl, of 1,3-propanediyl, 1,4-butanediyl, methylenedioxy (—O—CH₂—O—), and 1,2-ethylenedioxy (—O—CH₂—CH₂—O—), each of which is attached in the 2,3-position and are optionally mono- or polysubstituted by identical or different substituents slected from the group consisting of fluorine, chlorine, methyl, ethyl, n-propyl, i-propyl, and trifluoromethyl; represents pyridyl that is attached in the 2- or 4-position and is optionally mono- to tetrasubstituted by identical or different substituents selected from the group consisting of fluorine, chlorine, bromine, cyano, nitro, methyl, ethyl, methoxy, methylthio, hydroximinomethyl, hydroximinoethyl, methoximinomethyl, methoximinoethyl, and trifluoromethyl; represents pyrimidyl that is attached in the 2- or 4-position and is optionally mono- to trisubstituted by identical or different substituents selected from the group consisting of fluorine, chlorine, bromine, cyano, nitro, methyl, ethyl, methoxy, methylthio, hydroximinomethyl, hydroximinoethyl, methoximinomethyl, methoximinoethyl, and trifluoromethyl; represents thienyl that is attached in the 2- or 3-position and is optionally mono- to trisubstituted by identical or different substituents selected from the group consisting of fluorine, chlorine, bromine, cyano, nitro, methyl, ethyl, methoxy, methylthio, hydroximinomethyl, hydroximinoethyl, methoximinomethyl, methoximinoethyl, and trifluoromethyl; represents C₁-C₈-alkylamino or di-C₁-C₈-alkylamino; represents thiazolyl that is attached in the 2-, 4-, or 5-position and is optionally mono- or disubstituted by identical or different substituents selected from the group consisting of fluorine, chlorine, bromine, cyano, nitro, methyl, ethyl, methoxy, methylthio, hydroximinomethyl, hydroximinoethyl, methoximinomethyl, methoximinoethyl, and trifluoromethyl; or represents N-piperidinyl, N-tetrazolyl, N-pyrazolyl, N-imidazolyl, N-1,2,4-triazolyl, N-pyrrolyl, or N-morpholinyl, each of which is unsubstituted or mono- or polysubstituted by identical or different substituents selected from the group consisting of fluorine, chlorine, bromine, cyano, nitro, methyl, ethyl, methoxy, methylthio, hydroximinomethyl, hydroximinoethyl, methoximinomethyl, methoximinoethyl, and trifluoromethyl,
R⁴ represents H, Cl, F, CH₃, —CH(CH₃)₂, or cyclopropyl,
R⁵ represents H, Cl, F, CH₃, —CH(CH₃)₂, or cyclopropyl, and
X represents H, F, Cl, CN, or (C₁-C₄)-alkyl that is unsubstituted or substituted by one or more fluorine or chlorine atoms.

14. An imidazolopyrimidine of formula (I) according to claim 11 in which
R¹ represents H;
R² represents SiMe₃, SiMe₂Et, SiMe₂CHMe₂, SiMe₂CH₂CHMe₂, SiMe₂CH₂CMe₃, SiMe₂OCHMe₂, SiMe₂OCH₂CHMe₂, CH₂SiMe₃, CH₂SiMe₂Et, CH₂SiMe₂CHMe₂, CH₂SiMe₂CH₂CHMe, CH₂SiMe₂OMe, CH₂SiMe₂OCHMe₂, CH₂SiMe₂OCH₂CHMe₂, CHMeSiMe₃, CHMeSiMe₂OMe, (CH₂)₂SiMe₃, (CH₂)₂SiMe₂Et, (CH₂)₂SiMe₂CHMe₂, (CH₂)₂SiMe₂CMe₃, (CH₂)₂SiMe₂CH₂CHMe₂, (CH₂)₂SiMe₂CH₂CH₂Me, (CH₂)₂SiMe₂CH₂CMe₃, (CH₂)₂SiMe₂OCHMe₂, (CH₂)₂SiMe₂OCH₂CHMe₂, CHMeCH₂SiMe₃, CHMeCH₂SiMe₂Et, CHMeCH₂SiMe₂CH₂CH₂Me, CHMeCH₂SiMe₂CHMe₂, CHMeCH₂SiMe₂CMe₃, CHMeCH₂SiMe₂CH₂CHMe₂, CFMeCH₂SiMe₃, CHMeCH₂CH₂SiMe₂OMe, CHMeCH₂SiMe₂OCHMe₂, CHMeCH₂SiMe₂OCH₂CHMe₂, CH₂CHMeSiMe₃, CH₂CHMeSiMe₂Et, CH₂CHMeSiMe₂CHMe₂, CHMeCHMeSiMe₃, CMe₂CH₂SiMe₃, (CH₂)₃SiMe₃, (CH₂)₃SiMe₂Et, (CH₂)₃SiMe₂CHMe₂, (CH₂)₃SiMe₂CH₂CHMe₂, (CH₂)₃SiMe₂OMe, (CH₂)₃SiMe₂OCHMe₂, (CH₂)₃SiMe₂OCH₂CHMe₂, CHMeCH₂CH₂SiMe₃, CHMeCH₂CH₂SiMe₂Et, CHMeCH₂CH₂SiMe₂CHMe₂, CHMeCH₂CH₂CH₂SiMe₂OMe, CHMeCH₂CH₂SiMe₂OCHMe₂, CMe═CHSiMe₃, CH₂CH₂SiMe₂OMe, —C≡C—SiMe₃, —CH₂—C≡C—SiMe₃, or —CHMe—C≡C—SiMe₃,
R³ represents (C₁-C₆)-alkyl, (C₃-₆)-alkenyl, (C₃-C₆)-alkynyl, or (C₃-C₈)-cycloalkyl, where each such group is unsubstituted or substituted by one or more fluorine or chlorine atoms; represents 2,4- or 2,6-disubstituted phenyl, 2-substituted phenyl, or 2,4,6-trisubstituted phenyl; represents pyridyl that is attached in the 2- or 4-position and that is optionally mono- to tetrasubstituted by identical or different substituents from the group consisting of fluorine, chlorine, bromine, cyano, methyl, ethyl, methoxy, methylthio, hydroximinomethyl, hydroximinoethyl, methoximinomethyl, methoximinoethyl, and trifluoromethyl; or represents pyrimidyl that is attached in the 4-position and is optionally mono- to trisubstituted by identical or different substituents selected from the group consisting of fluorine, chlorine, bromine, cyano, methyl, ethyl, methoxy, methylthio, hydroximinomethyl, hydroximinoethyl, methoximinomethyl, methoximinoethyl, and trifluoromethyl,
R⁴ represents H, —CH₃, —CH(CH₃)₂, Cl, or cyclopropyl,
R⁵ represents H, —CH₃, —CH(CH₃)₂, Cl or cyclopropyl, and
X represents fluorine, chlorine, (C₁-C₇)-alkyl, or (C₁-C₃)-haloalkyl.

15. A process for preparing imidazolopyrimidines of the formula (I) according to claim 11 comprising
(a) for imidazolopyrimidines of formula (I-1) 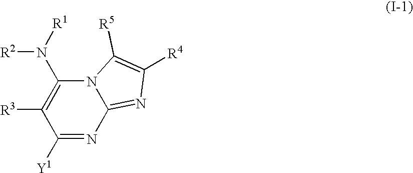
in which
R¹, R², R³, R⁴, and R⁵ are as defined for formula (I) of claim 11, and
Y¹ represents halogen,
reacting a haloimidazolopyrimidine of formula (II-1) 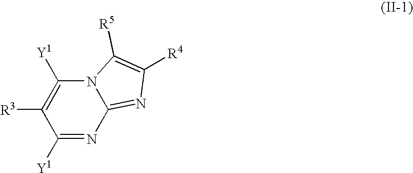
in which
R³, R⁴, and R⁵ are as defined for formula (I) of claim 11, and
Y¹ represents halogen,
with an amine of formula (III) 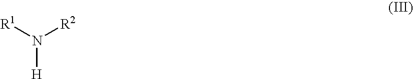
in which R¹ and R² are as defined for formula (I) of claim 11, optionally in the presence of a diluent, optionally in the presence of an acid acceptor, and optionally in the presence of a catalyst,
or
(b) for imidazolopyrimidines of formula (I-2) 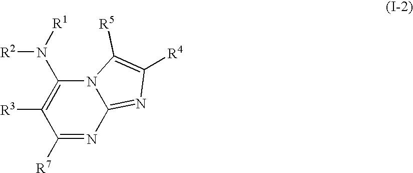
in which
R¹, R², R³, R⁴, and R⁵ are as defined for formula (I) of claim 11, and
R⁷ represents optionally substituted alkyl or optionally substituted phenyl,
reacting a haloimidazolopyrimidine of formula (II-2) 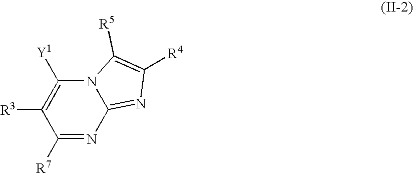
in which
R³, R⁴, and R⁵ are as defined for formula (I) of claim 11,
Y¹ represents halogen, and
R⁷ represents optionally substituted alkyl or optionally substituted phenyl,
with an amine of formula (III) 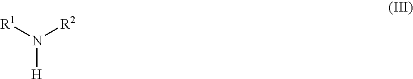
in which R¹ and R² are as defined for formula (I) of claim 11, optionally in the presence of a diluent, optionally in the presence of an acid acceptor, and optionally in the presence of a catalyst,
or
(c) for imidazolopyrimidines of formula (I-3) 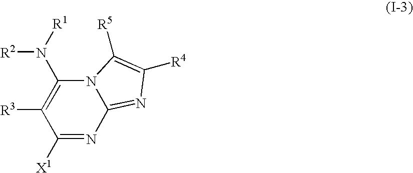
in which
R¹, R², R³, R⁴, and R⁵ are as defined for formula (I) of claim 11, and
X¹ represents cyano or optionally substituted alkoxy,
reacting an imidazolopyrimidine of formula (I-1) 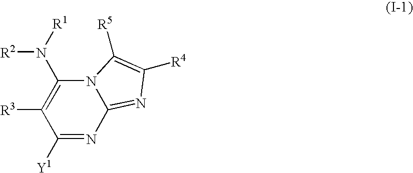
in which
R¹, R², R³, R⁴, and R⁵ are as defined for formula (I) of claim 11, and
Y¹ represents halogen,
with a compound of formula (IX),
M-X¹ (IX)
in which
M represents ammonium, tetraalkylammonium, an alkali metal cation, or an alkaline earth metal cation, and
X¹represents cyano, alkoxy, or substituted alkoxy.

16. A composition for controlling unwanted microorganisms comprises one or more imidazolopyrimidines of formula (I) according to claim 11 and one or more extenders and/or surfactants.

17. A composition according to claim 16 additionally comprising one or more further agrochemically active compounds.

18. A method for controlling unwanted microorganisms comprising applying an effective amount of an imidazolopyrimidine of formula (I) according to claim 11 to the unwanted microorganisms and/or their habitats.

19. A process for preparing a composition for controlling unwanted microorganisms comprising mixing one or more imidazolopyrimidines of formula (I) according to claim 11 with one or more extenders and/or surfactants.
